(19) United States
(12) Patent Application Publication
(10) Pub. No.: US 2019/0060129 A1
(43) Pub. Date: Feb. 28, 2019

(54) COLD THERAPY CAST BOOT

(71) Applicant: Christopher Mark LU, Mississauga (CA)

(72) Inventor: Christopher Mark LU, Mississauga (CA)

(21) Appl. No.: 16/108,776

(22) Filed: Aug. 22, 2018

Related U.S. Application Data
(60) Provisional application No. 62/548,471, filed on Aug. 22, 2017.

(51) Int. Cl. A61F 13/04; A61F 5/01

(52) U.S. Cl. CPC A61F 13/046; A61F 13/043; A61F 13/045; A61F 5/0111; A61F 5/0195

(57) ABSTRACT

A cast boot and removable integrated cold therapy attachment is provided for promoting healing, recovery and pain relief, following lower limb fracture or surgery. The cast boot has at least one channel for drainage of condensation or liquid from the interior of the cast boot. The cast boot is configured to support a cold therapy attachment. The cold therapy attachment further has one or more perforated holes for drainage of condensation or liquid. The cast boot has adjustable straps for allowing removable and positioning of the cold therapy attachment inside the cast boot.

### CROSS-REFERENCE TO RELATED APPLICATIONS

The present disclosure claims priority from U.S. provisional patent application No. 62/548,471, filed Aug. 22, 2017, the entirety of which is hereby incorporated by reference.

### FIELD

The present disclosure relates generally to an immobilization device and method for post-operative and post-injury healing. Particularly, the present disclosure relates to an integrated cast boot and cold therapy device and method.

### BACKGROUND

Injuries to the musculoskeletal system (e.g., bones, joints, muscles, ligaments, tendons) may require surgery. Post-operative pain may be significant for two days after surgery, but may last up for up to at least two weeks after surgery, depending on the nature and extent of the injury. Pain may last longer than two weeks, for example, for several months subsequent to surgery.

The injury should be immobilized during the recovery period, and the patient may be required to wear a suitable immobilization device to protect the fractured area and promote healing in the wounded area. Current treatment methods for fractures include splints, plaster casts, fiberglass casts, cast-braces, cast boots or other devices to immobilize the fracture or surgical area while the bone and soft-tissue is healing.

The patient further may be required to apply a cold therapy device to the wounded area, and may apply the cold therapy in conjunction with the immobilization device. The patient generally should remove the immobilization device as little as possible to prevent bacteria and germs from entering into the wounded area. However, non-removal of the immobilization device may cause condensation or fluid buildup. Especially when the cold therapy device is further encased in a cast, cast boot or other device.

### SUMMARY

In various examples disclosed herein, the present disclosure describes a cast boot configured to support a removable integrated cold therapy attachment for promoting healing and recovery, for example, for a foot or lower limb fracture. The cast boot further includes at least one channel for drainage of condensation or liquid from the interior of the cast boot. The cold therapy attachment further includes one or more perforated holes for drainage of condensation or liquid.

In one example aspect there is provided a cast boot that includes a bottom portion configured for embracing a foot. The cast boot also includes at least one channel configured for drainage of condensation from an interior portion of the cast boot.

### BRIEF DESCRIPTION OF THE DRAWINGS

Reference will now be made, by way of example, to the accompanying drawings which show example embodiments of the present application, and in which:

FIG. 1 is a side view of an example boot with a cold therapy attachment inserted therein;

FIG. 2 is an opposed side view of the boot and cold therapy attachment of FIG. 1;

FIG. 3 is a front view of an example cold therapy attachment;

FIG. 4 is an image of a side view of an example cold therapy attachment and liner for insertion into a boot;

FIG. 5 is an image of a side view of an example boot without the cold therapy attachment and liner;

FIG. 6 is an image of the example cold therapy attachment and liner of FIG. 4 inserted into the boot of FIG. 5;

FIG. 7 is an image of a front view of the example boot of FIG. 5 opened up;

FIG. 8 is an image of a front view of the example boot of FIG. 5 with the example cold therapy attachment and liner of FIG. 4 inserted;

FIG. 9 is an image of an opposed side view of the example cold therapy attachment and liner inserted into the example boot of FIG. 5;

FIG. 10 is an image of a disassembled side view of a boot, liner and cold therapy attachment;

FIG. 11 is an image of a side view of an assembled cold therapy attachment and liner and the example boot of FIG. 5;

FIG. 12 is an opposed side view of the assembled boot, cold therapy attachment and liner of FIG. 9;

FIG. 13 is a front view of an example boot and inserted cold therapy attachment of FIG. 3;

FIG. 14 is a front view of the example boot of FIG. 13 opened up and without cold therapy attachment and liner; and

FIG. 15 is side view of the example boot of FIG. 13 with a removable collecting device for collecting condensation.

Similar reference numerals may have been used in different figures to denote similar components.

### DESCRIPTION OF EXAMPLE EMBODIMENTS

The following disclosure provides an example cold therapy cast boot with an integrated cold therapy.

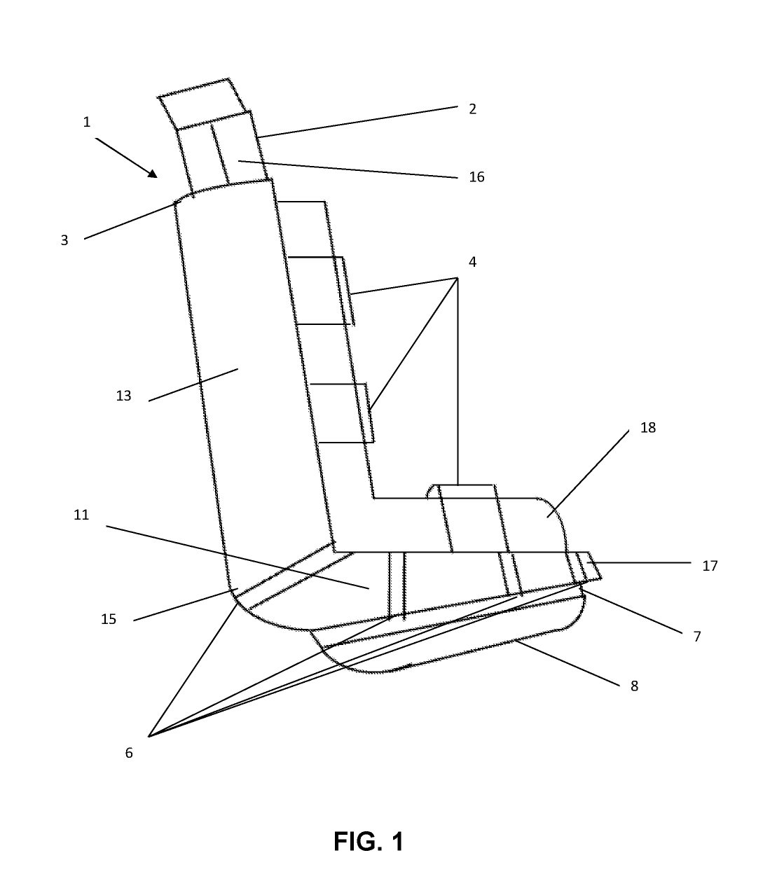

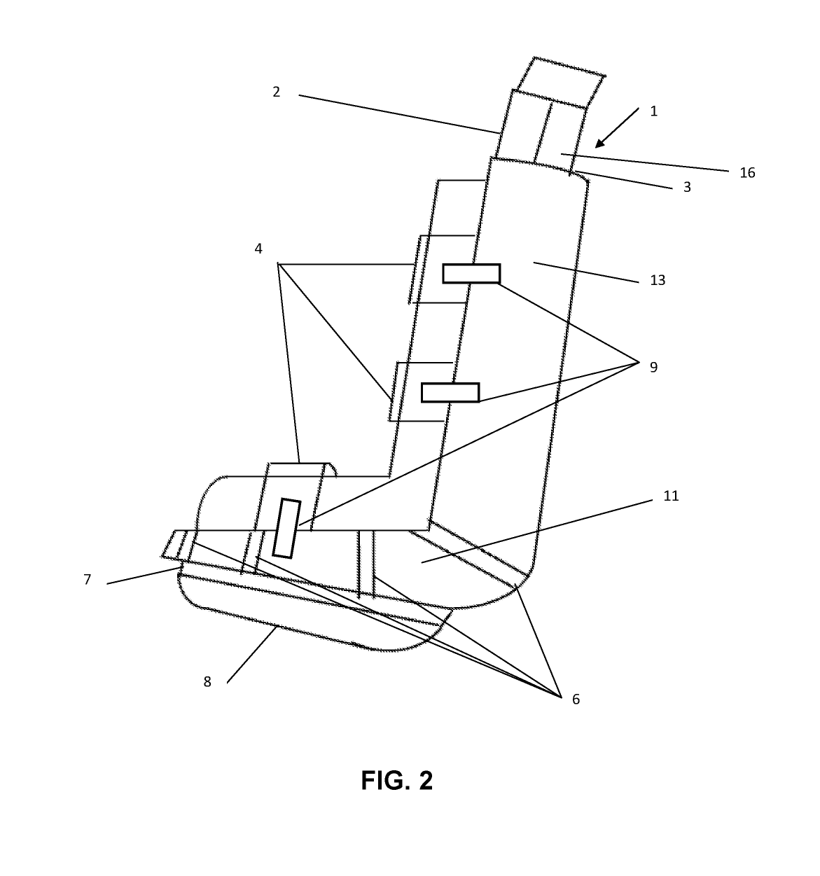

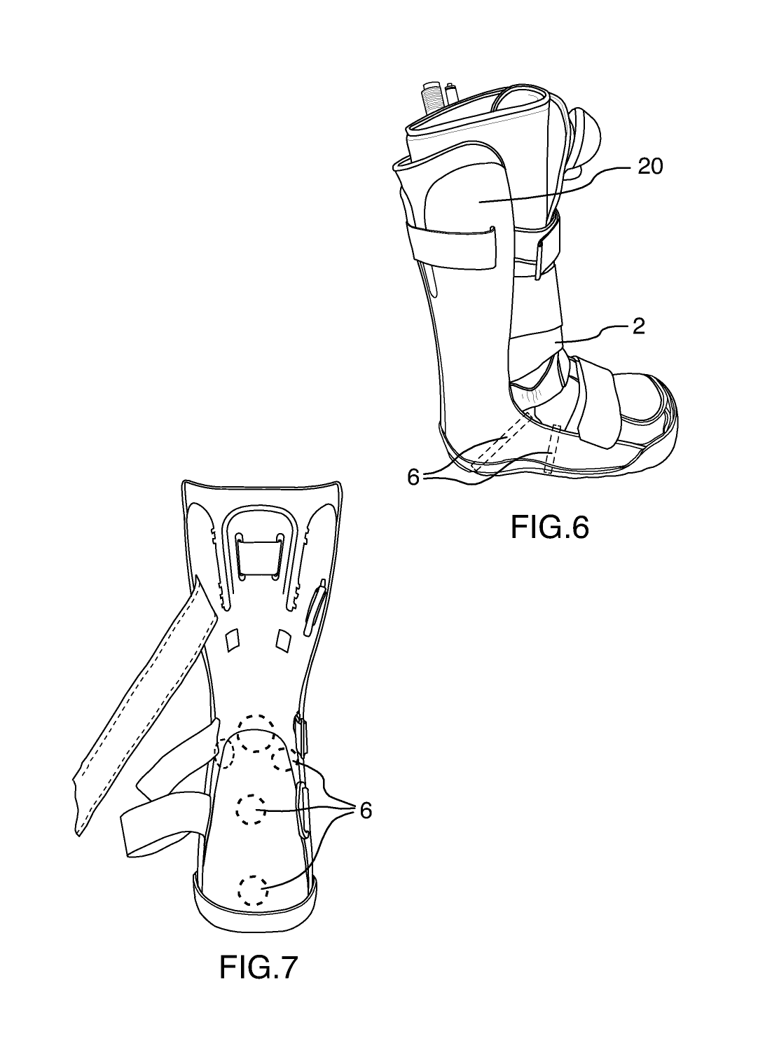

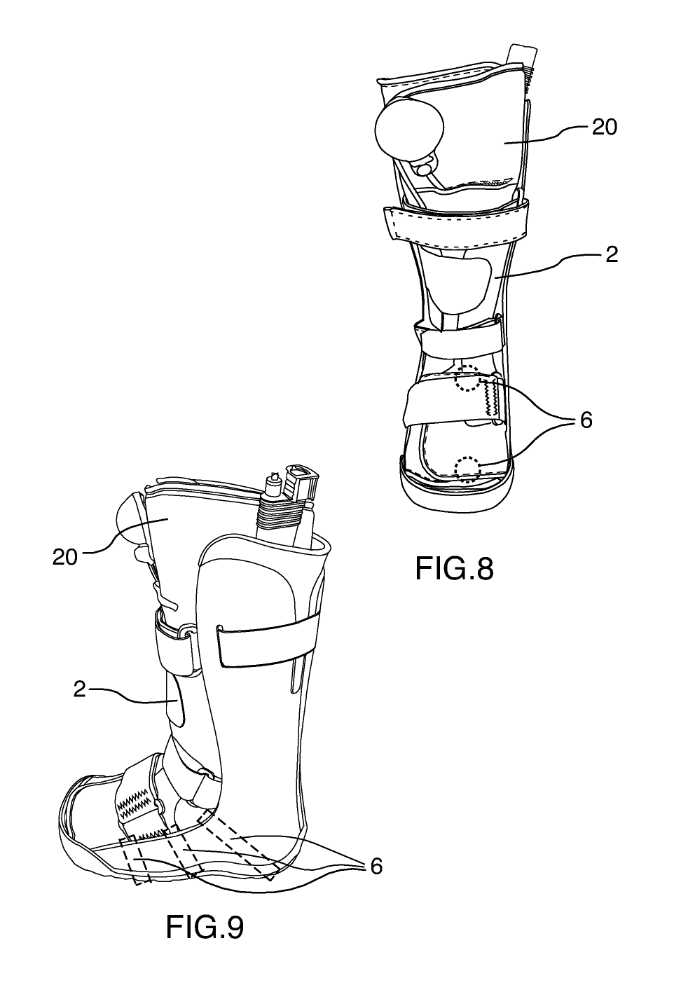

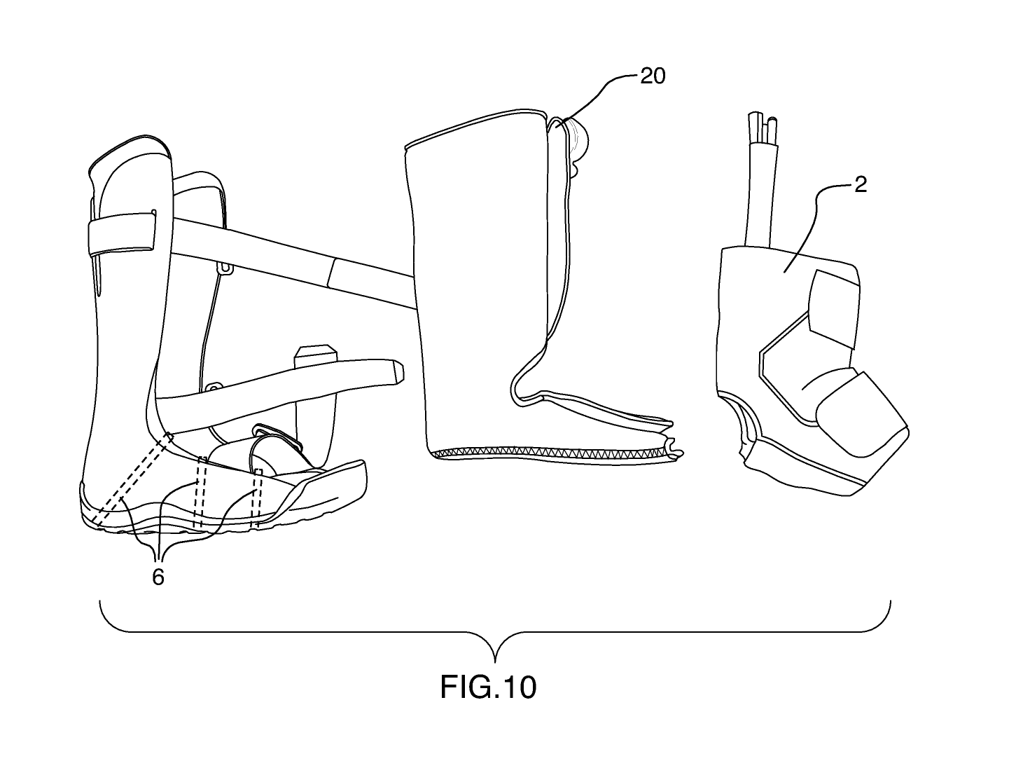

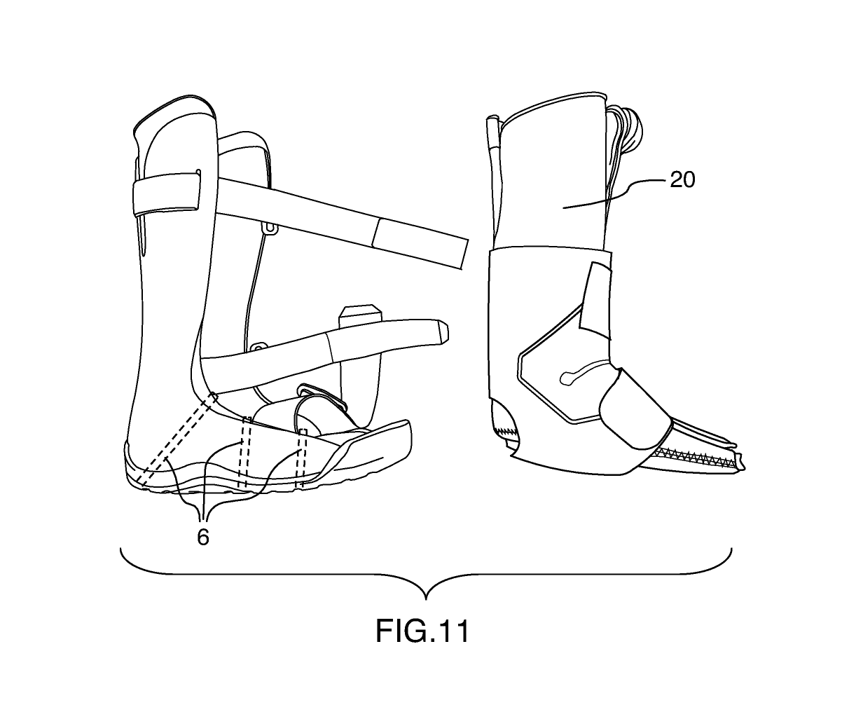

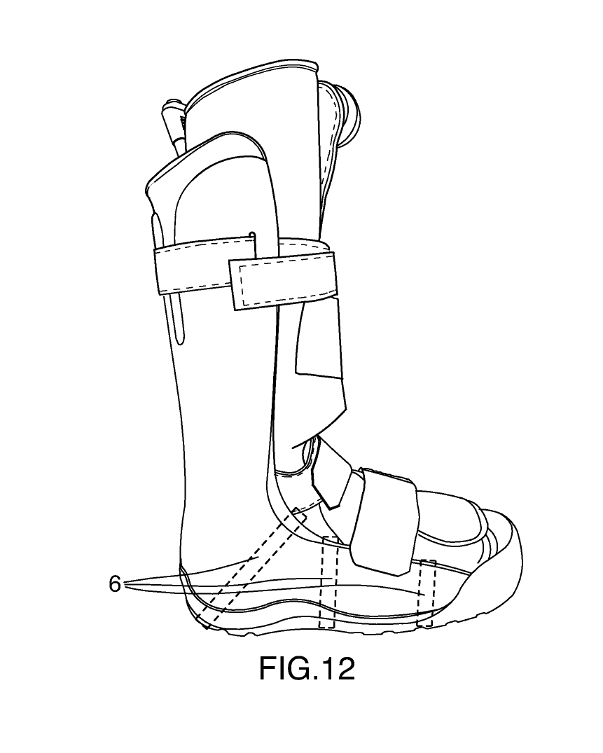

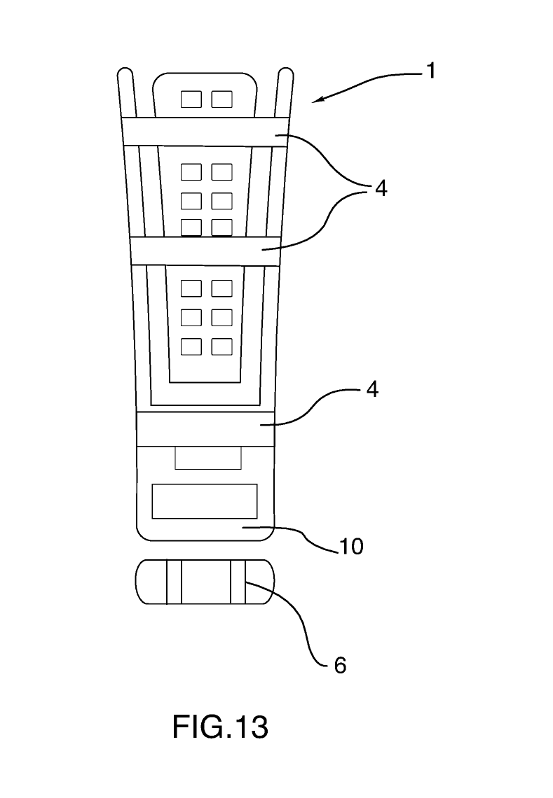

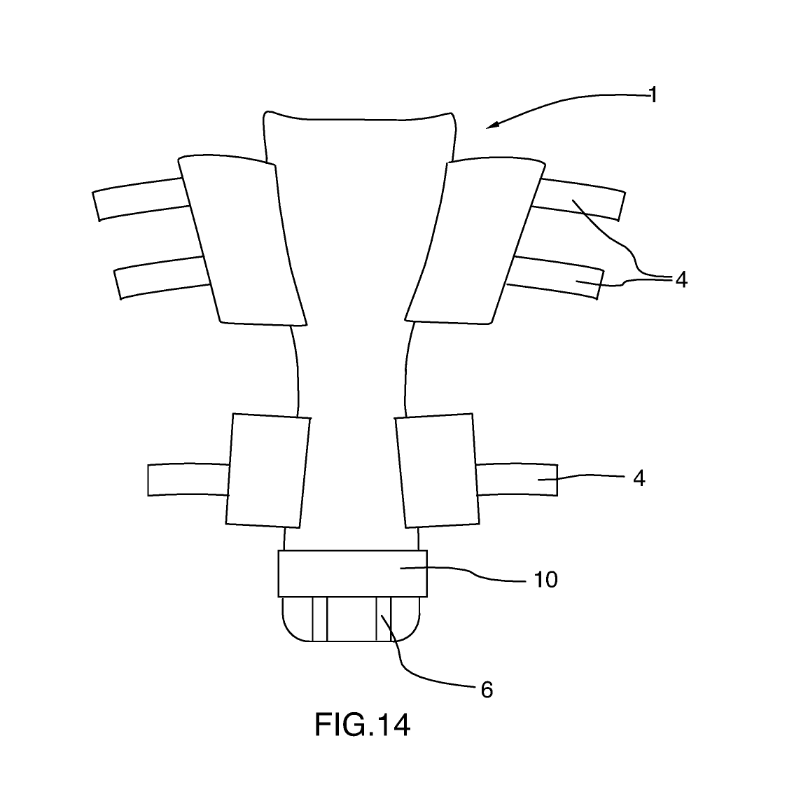

FIGS. 1-2, and 5-15 illustrate an example embodiment of a cold therapy cast boot 1. Cold therapy attachment 2 is insertable into cold therapy cast boot 1 and removable therefrom.

Cast boot 1 may be shaped to fit around and enclose or embrace a foot and at least part of a lower limb of a patient. In an example embodiment, cast boot 1 protects and embraces the foot and lower limb while permitting a patient to walk. Cast boot 1 may be configured to conform to or generally follow the shape of the patient's foot and lower limb while allowing sufficient space for the insertion of cold therapy attachment 2 along with the foot and lower limb.

Cast boot 1 may include a bottom portion 11 adapted to support or embrace the foot. Cast boot 1 may further include an elongated portion 13 extending upwardly from the bottom portion 11 configured for supporting and embracing at least part of a lower leg of a patient. The elongated portion 13 of cast boot 1 may be of various heights. For example, a shorter version (e.g. 1 cm) of elongated portion 13 may be used for a foot injury or elongated portion 13 may further be absent. A longer version of elongated portion 13 (e.g. 10-50 cm) may be used for ankle or tibia injuries. The length of elongated portion 13 provided is not meant to be limiting, and additional lengths may be used.

Cast boot 1 may also include a heel portion 15 for enclosing the heel and/or a toe portion 18 for enclosing the toe.

Cast boot 1 may be configured to support or embrace the foot and at least part of the lower leg of a patient while moving, for example while walking, stretching or mildly exercising. Cast boot 1 further may be configured to support the foot and at least part of the lower leg of a patient while the patient is resting and/or immobile or relatively immobile. Generally, the cast boot 1 may be configured such that the foot and at least part of the lower leg is immobilized. That is, the bottom portion 11 and the elongated portion 13 may be sufficiently rigid such that when the foot and lower leg is supported therein, movement of the foot and lower leg is restricted.

Cast boot 1 may include a rigid or semi-rigid material or structure configured for providing support to the foot and/or at least part of a lower limb of the patient. Cast boot 1 may further include a semi-flexible material. Generally, cast boot 1 may be fabricated using materials similar to those of conventional cast boots commonly used for immobilization of a patient's foot.

Cast boot 1 may include one or more straps 4. At least one of straps 4 allow for the size and/or volume of the cast boot 1 to be adjustable according to the size of the lower limb of the patient. Further, the patient may prefer cast boot 1 to fit loosely and at least one of straps 4 may be adjusted accordingly. Alternatively, the patient may prefer cast boot 1 to fit tightly and at least one of straps 4 can be adjusted accordingly.

One or more straps 4 may be connectable to the front of cast boot 1 and may be adjustable and open-and-closeable. One or more straps 4 may be loosened to allow for cast boot 1 to be opened along elongated portion 13 and a top of bottom portion 11.

One or more straps 4 may allow for the cast boot 1 to be opened for insertion and positioning of cold therapy attachment 2. Cold therapy attachment 2 may be positioned to be as close to the wounded or fractured area of the limb or foot as possible when the cast boot 1 is worn by the patient. Cold therapy attachment 2 may be inserted into cast boot 1 and positioned along an inner portion of elongated portion 13. Cold therapy attachment 2 may be inserted into cast boot 1 and positioned along an inner portion of bottom portion 11. Alternatively, cold therapy attachment 2 may be inserted and positioned along a combination of elongated portion 13 and bottom portion 11.

Cold therapy attachment 2 may be inserted into cast boot 1 before or after insertion of the foot and/or limb of the patient.

The foot of a patient and/or lower limb is insertable into cast boot 1. Straps 4 secure the foot of the patient and/or lower limb in place and help prevent the foot and/or lower limb from moving. Once the foot and/or lower limb have been inserted, the straps 4 can be tightened to secure the foot in place.

Straps 4 may include fasteners 9, for example for allowing at least one end of straps 4 to be removeably secured to the boot 4 so that the patient can insert and remove the foot and/or cold therapy attachment 2. Fasteners 9 may comprise Velcro™ fasteners and/or clips. Other attachment means are also possible. The patient can release the straps 4 upon inserting or removing the foot and/or lower limb and/or cold therapy attachment 2.

Bottom portion 11 of cast boot 1 further includes a walking heel 8, which may be detachable. Walking heel 8 may be configurable to attach or detach from connector element 7, for example by sliding on and off of connector element 7 or through any other suitable removable attachment means. Connector element 7 may be connected to or integral with a bottom portion of boot 4.

Walking heel 8 may not need to be removed for drainage of condensation. Further, walking heel 8 may not be removable or detachable. In some examples, a regular non-removal heel may be used in place of walking heel 8.

In an example embodiment, cast boot 1 includes at least one drainage channel 6 for draining excess moisture, condensation or liquid buildup from the interior of cast boot 1. In FIGS. 5-12, the channels 6 are illustrated in dotted lines, to indicate that they are located within the boot 1 and would not necessarily been seen from the views shown.

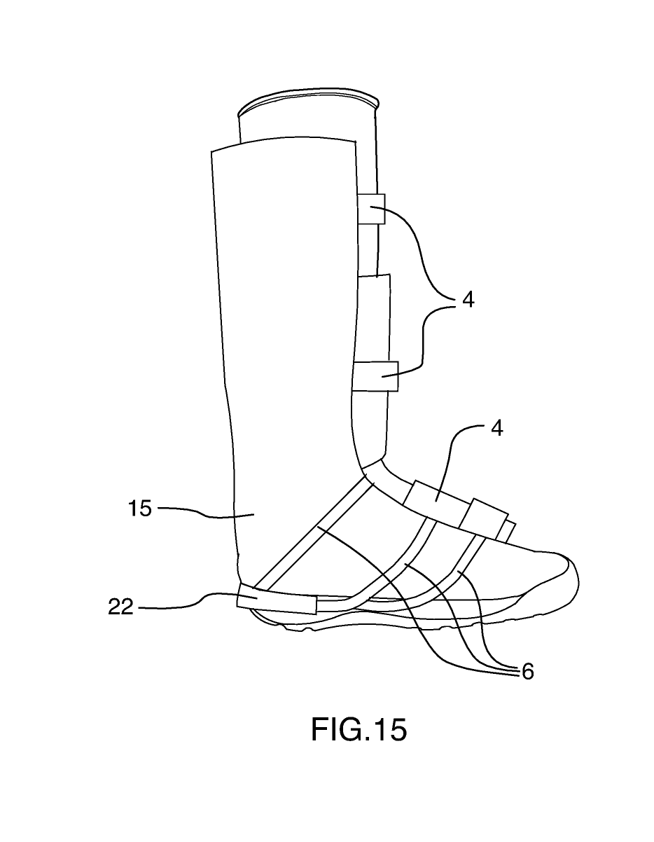

Condensation build up tends to occur in an immobilization device due to cooling of air by a liner, in the case where the liner provides cold therapy or is otherwise cooler than the interior air. Moisture or condensation build up may present problems to a user (e.g., discomfort and/or risk of infection), and it is desirable to be able to drain the condensation from the immobilization device. Patients may sometimes remove the immobilization device in order to remove condensation. However, this is not desirable as a fractured or wounded area should be immobilized and the immobilization device should ideally be left on for as long as required for a wound to heal. The presence of one or more drainage channels 6 may enable removal of at least some of this moisture, without requiring removal of the immobilization device. A removable device (described further below with reference to FIG. 15) may be provided (e.g., at the heel) to help collect the condensation and prevent dripping on the ground.

At least one drainage channel 6 may be positioned at the heel portion 15 of cast boot 1. At least one drainage channel 6 may be positioned at a bottom portion 11 of cast boot 1 and/or on the bottom portion 11 at a toe portion 17 of cast boot 1. Further, at least one drainage channel 6 may be positioned along the elongated portion 13. In an example embodiment, a drainage channel 6 is positioned at the heel portion 15, two drainage channels 6 are positioned at the bottom portion 11, and one drainage channel 6 is positioned at the toe portion 17 of cast boot 1. Drainage channels 6 may be positioned at heel portion 15 and toe portion 17 or at extreme ends of boot 4 in order to avoid buildup at dead ends of the boot 4. Additional drainage channels 6 may be provided midway between the extreme ends, for more effective drainage. Alternate designs, arrangements and numbers of drainage channels 6 are also possible.

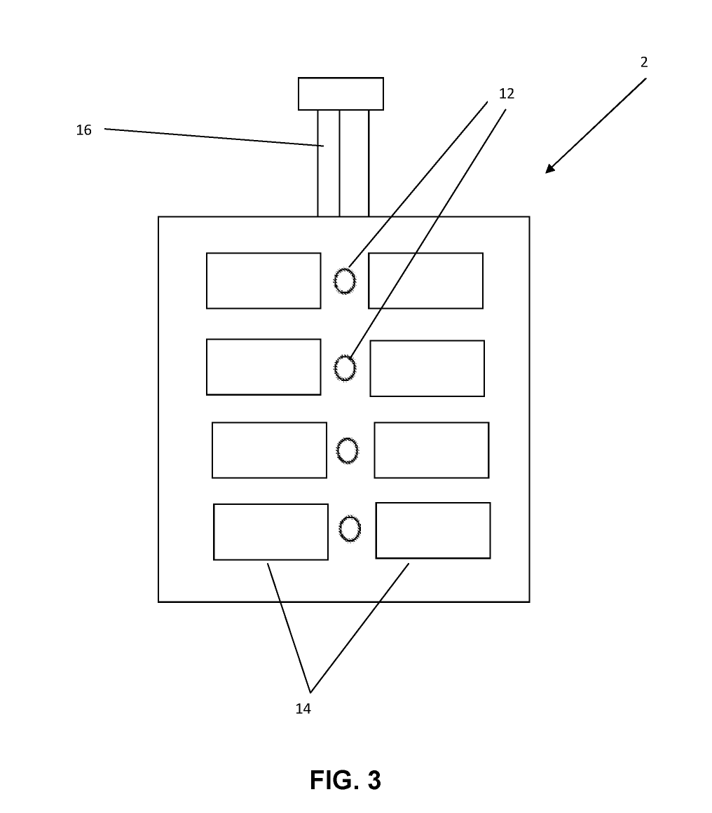

In an example embodiment, at least one drainage channel 6 may be aligned with (e.g., immediately underneath) at least one perforation 12 (described below with reference to FIG. 3) of cold therapy attachment 2, when the cold therapy attachment 2 is properly inserted into the cast boot 1. In some embodiments, at least one drainage channel 6 may not be aligned with at least one perforation 12 of cold therapy attachment 2. In some examples the number of drainage channels 6 corresponds to the number of perforated holes and the drainage channels 6 all align with the perforations 12.

With reference to FIG. 3, in an example embodiment, cold therapy attachment 2 includes one or more perforations 12 that, together with the at least one drainage channel 6, allow for drainage of condensation or liquid build up from the interior of the cast boot 1 when the cold therapy attachment 2 is inserted. In an example embodiment, a plurality of perforations 12 are incorporated into cold therapy attachment 2 allowing for the draining of condensation or liquid.

Cold therapy attachment 2 further may include at least one cold therapy member 14 for applying to and promoting recovery in the fractured or wounded area.

Cold therapy attachment 2 may be inserted and positioned inside cast boot 1. Upon insertion of a foot and/or lower limb of a patient into cast boot 1, cold therapy member 14 may come into direct contact or close contact with the fractured or wounded area of the foot and/or lower limb. Alternatively, cold therapy attachment 2 may first be applied to the fractured/wounded area of the patient, and the foot and/or lower limb together with the cold therapy attachment 2 may be inserted into cast boot 1.

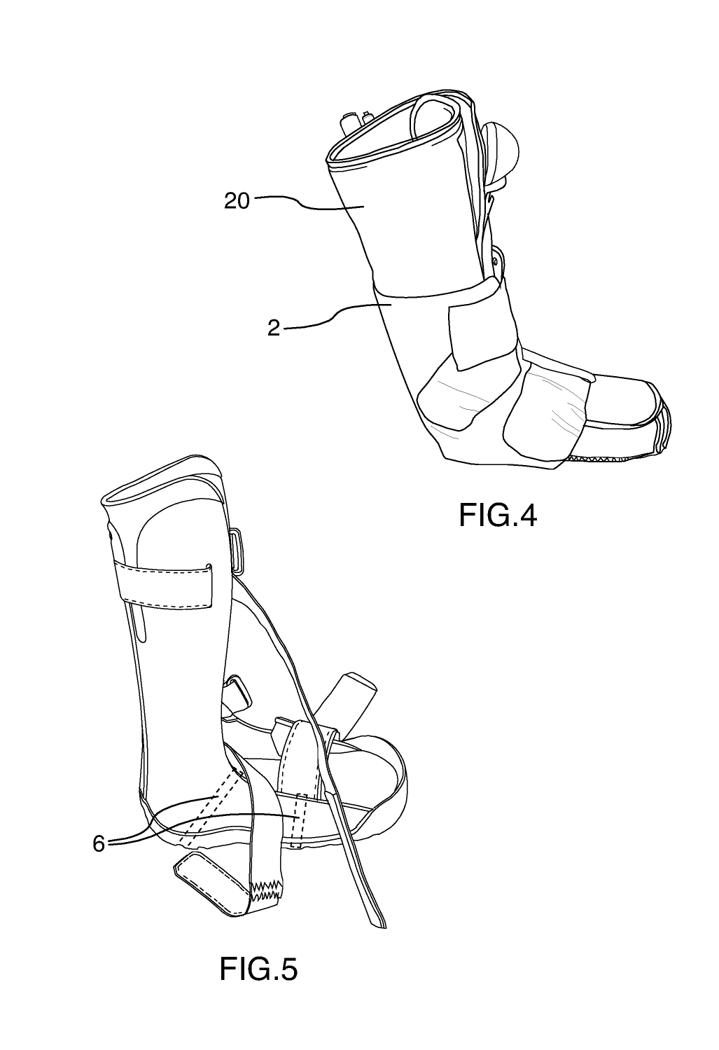

With reference to FIG. 4, a boot liner 20 may be used with the cold therapy attachment 2 and inserted together with the cold therapy attachment 2 into the boot 1. The liner 20 may comprise padding for providing cushioning and padding to the foot of the patient. The liner 20 may provide other benefits, such as having anti-odor, anti-septic properties, and non-absorbent properties. In some examples, the liner 20 may be an antimicrobial liner. The cold therapy attachment 2 may be positioned to embrace the boot liner 20. In some examples, the cold therapy attachment 2 may be inserted into the boot 1 without using a liner 20.

Cold therapy attachment 2 may come in a variety of shapes and sizes. For example, cold therapy attachment 2 may be substantially square, rectangular, oval or circular. Further, the number of perforations 12 may vary, for example, from 1 hole to 50 holes or more.

In an example embodiment, perforations 12 on cold therapy attachment 2 allow for drainage of condensation or liquid that may collect in the interior of cast boot 1. The liquid or condensation is further drained through at least one drainage channel 6 of cast boot 1. In an example embodiment, at least one perforation 12 of cold therapy attachment 2 is positioned adjacent to drainage channel 6. In some example embodiments, at least one perforation 12 is not positioned adjacent to drainage channel 6. Due to the forces of gravity and kinetic forces from the patient moving, liquid or condensation can flow through at least one perforation 12 and through at least one drainage channel 6 even if at least one perforation 12 is not positioned adjacent to at least one drainage channel 6.

Cold therapy attachment 2 further comprises a handle 16, which is positionable through an opening 3 of cold therapy cast boot 2, allowing for easy removal of cold therapy attachment 2.

As mentioned above, the cold therapy attachment 2 may be used without a separate liner 20. For example, the cold therapy attachment 2 may be itself serve as a liner (and may include properties such as being anti-odor, anti-septic, non-absorbent and/or antimicrobial, for example) that may be interchangeable, disposable and/or reusable, allowing for switching with a standard liner 20 or a liner containing an antiseptic, or for hygienic reasons, for example to avoid a bad smell. Further, cold therapy attachment 2 may itself provide antiseptic properties, for example having an overlying fabric with antiseptic and hygienic properties.

With reference to FIG. 15, cast boot 1 may further include a collecting device 22 for collecting excess condensation. Collecting device 22 may be a cup or similar device. Collecting device 22 may be transparent or may be opaque. Collecting device 22 may be removeable or may be secured to cast boot 1. If collecting device 22 is secured to cast boot 1, collecting device 22 may be openable to allow collected condensation to be poured out. Collecting device 22 may be positioned at heel portion 15, for example. Collecting device 22 may further be positioned at toe portion 18, or another area of the cast boot 1 suitable for collecting excess condensation. In some examples, there may be more than one collecting device 22 provided at different locations.

Alternative shapes and embodiments of cast boot 1 are also possible. For example, cast boot 1 may not include toe portion 18, which may be dependent on the requirements of the patient. Cast boot 1 may not include a heel portion 15, which may be dependent on the requirements of a patient.

An example study was performed using the example cast boot 1 with cold therapy attachment 2, as shown in FIG. 12. In the example study, patients undergoing ankle arthroscopy and brostrum/ligament repair were divided into two cohorts. Cohort 1 was treated using a conventional cast boot. Cohort 2 was treated using the disclosed cast boot with cold therapy attachment. The patients were questioned in regards to pain medication used at 2 weeks post-op. The study found that patients using the disclosed cast boot with cold therapy attachment used less Percocet™ at 2 weeks post-op. Specifically, Cohort 1 (treated using conventional cast boot without cold therapy attachment) used, on average, 48 tablets of Percocet at 2 weeks post-op; whereas Cohort 2 (treated using the disclosed cast boot with cold therapy attachment) used, on average, 17 tablets of Percocet at 2 weeks post-op. This demonstrates that patients treated using an embodiment of the present disclosure benefited from less reliance on pain medication.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

1. A cast boot comprising:
a bottom portion configured for supporting and immobilizing a foot;
at least one channel in the bottom portion configured for drainage of moisture from an interior portion of the cast boot, wherein the cast boot is configured to support a cold therapy attachment that is removeably insertable into the cast boot.

2. The cast boot of claim 1, wherein the at least one channel is positioned at a heel portion of the cast boot.

3. The cast boot of claim 1, wherein the at least one channel is positioned at a front portion of the bottom portion of the cast boot.

4. The cast boot of claim 1, further comprising a walking heel removably securable to the bottom portion of the cast boot.

5. The cast boot of claim 1, further comprising the cold therapy attachment.

6. The cast boot of claim 5, wherein the cold therapy attachment defining at least one perforation.

7. The cast boot of claim 6, wherein the at least one channel is located in the bottom portion to be aligned with the at least one perforation, when the cold therapy attachment is inserted into the cast boot.

8. The cast boot of claim 5, wherein the cold therapy attachment comprises a handle.

9. The cast boot of claim 5, further comprising a liner.

10. The cast boot of claim 9, wherein the liner has at least one property selected from: anti-odor, anti-septic, non-absorbent, or antimicrobial.

11. The cast boot of claim 9, wherein the liner is integrated with the cold therapy attachment.

12. The cast boot of claim 1, further comprising at least one strap.

13. The cast boot of claim 1, further comprising an elongated portion configured to be worn over at least a portion of a lower limb, the elongated portion extending from the bottom portion.

14. The cast boot of claim 1 further comprising a removable collecting device for collecting the moisture.

15. The cast boot of claim 14 wherein the removable collecting device is positioned at a heel portion of the cast boot.

16. The cast boot of claim 14 wherein the removable collecting device is a cup device.
